(19) United States
(12) Patent Application Publication
(10) Pub. No.: US 2006/0205768 A1
(43) Pub. Date: Sep. 14, 2006

(54) Tropane derivatives and their use as ace inhibitors

(76) Inventors: Olivier Bezencon, Riehen (CH); Daniel Bur, Allschwil (CH); Walter Fischli, Allschwil (CH); Lubos Remen, Allschwil (CH); Sylvia Richard-Bildstein, Dietwiller (FR); Thomas Weller, Binningen (CH); Thierry Sifferlen, Guewenheim (FR)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/554,704

(22) PCT Filed: Apr. 26, 2004
(86) PCT No.: PCT/EP04/04375

§ 371 Date: Oct. 27, 2005

(30) Foreign Application Priority Data
Apr. 30, 2003 (WO) PCT/EP03/04490

(51) Int. Cl. A61K 31/46; C07D 451/02

(52) U.S. Cl. 514/304; 546/126

(57) ABSTRACT

The invention relates to novel tropane derivatives and related compounds and their use as active ingredients in the preparation of pharmaceutical compositions. The invention also concerns related aspects including processes for the preparation of the compounds, pharmaceutical compositions containing one or more of those compounds and especially their use as inhibitors of renin.

The invention relates to novel compounds of the general formula I. The invention also concerns related aspects including processes for the preparation of the compounds, pharmaceutical compositions containing one or more compounds of formula I and especially their use as renin inhibitors in cardiovascular events and renal insufficiency. Furthermore, these compounds can be regarded as inhibitors of other aspartyl proteases and might therefore be useful as inhibitors of plasmepsins to treat malaria and as inhibitors of Candida albicans secreted aspartyl proteases to treat fungal infections.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes to Ang II, whose concentration is dramatically increased by the blockade of AT₁ receptors. This may raise serious questions regarding the safety and efficacy profile of AT₁ receptor antagonists. In summary, renin inhibitors are not only expected to be different from ACE inhibitors and AT₁ blockers with regard to safety, but more importantly also with regard to their efficacy to block the RAS.

Only limited clinical experience (Azizi M. et al., J. Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are missing and sought. Recently, the first non-peptide renin inhibitors were described which show high in vitro activity (Oefner C. et al., Chem. Biol., 1999, 6, 127; Patent Application WO97/09311; Märki H. P. et al., Il Farmaco, 2001, 56, 21). However, the development status of these compounds is not known.

The present invention relates to the identification of renin inhibitors of a non-peptidic nature and of low molecular weight. Orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis are described.

The present invention describes non-peptidic renin inhibitors.

In particular, the present invention relates to novel compounds of the general formula I, 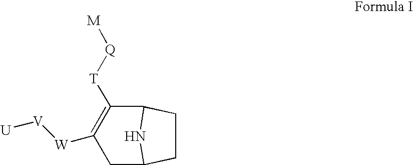
wherein
W is a six-membered, non benzofused, phenyl or heteroaryl ring, substituted by V in meta or para position;
V represents a bond; —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂-B-CH₂—; —CH₂-A-CH₂—CH₂—B—; —CH₂—CH₂—CH₂-A-CH₂—CH₂—; —CH₂—CH₂—CH₂—CH₂-A-CH₂—; -A- CH₂—CH₂—B—CH₂—CH₂—; —CH₂-A-CH₂—CH₂—B—CH₂—; —CH₂-A-CH₂—CH₂—CH₂—B—; —CH₂—CH₂-A-CH₂—CH₂—B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O—; or —O—C(CH₂CH₂)—CH₂—O—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; lower alkenylene;
M represents hydrogen; cycloalkyl; aryl; heterocyclyl; heteroaryl;
R¹ represents hydrogen; lower alkyl; lower alkenyl; lower alkinyl; cycloalkyl; aryl; cycloalkyl-lower alkyl;
p is the integer 1, 2, 3 or 4;
r is the integer 3, 4, 5, or 6;
s is the integer 2, 3, 4, or 5;
t is the integer 1, 2, 3, or 4;
u is the integer 1, 2, or 3;
v is the integer 2, 3, or 4;
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

In the definitions of general formula I—if not otherwise stated—the term lower alkyl, alone or in combination with other groups, means saturated, straight and branched chain groups with one to seven carbon atoms, preferably one to four carbon atoms that can be optionally substituted by halogens. Examples of lower alkyl groups are methyl, ethyl, n-propyl, iso-propyl, n-butyl, iso-butyl, sec-butyl, tert-butyl, pentyl, hexyl and heptyl. The methyl, ethyl nad isopropyl groups are preferred.

The term lower alkoxy refers to a R—O group, wherein R is a lower alkyl. Examples of lower alkoxy groups are methoxy, ethoxy, propoxy, iso-propoxy, iso-butoxy, sec-butoxy and tert-butoxy.

The term lower alkenyl, alone or in combination with other groups, means straight and branched chain groups comprising an olefinic bond and consisting of two to seven carbon atoms, preferably two to four carbon atoms, that can be optionally substituted by halogens. Examples of lower alkenyl are vinyl, propenyl or butenyl.

The term lower alkinyl, alone or in combination with other groups, means straight and branched chain groups comprising a triple bond and consisting of two to seven carbon atoms, preferably two to four carbon atoms, that can be optionally substituted by halogens. Examples of lower alkinyl are ethinyl, propinyl or butinyl.

The term lower alkylene, alone or in combination with other groups, means straight and branched divalent chain groups with one to seven carbon atoms, preferably one to four carbon atoms, that can be optionally substituted by halogens. Examples of lower alkylene are ethylene, propylene or butylene.

The term lower alkenylene, alone or in combination with other groups, means straight and branched divalent chain groups comprising an olefinic bond and consisting of two to seven carbon atoms, preferably two to four carbon atoms, that can be optionally substituted by halogens. Examples of lower alkenylene are vinylene, propenylene and butenylene.

The term lower alkylenedioxy, refers to a lower alkylene substituted at each end by an oxygen atom. Examples of lower alkylenedioxy groups are preferably methylenedioxy and ethylenedioxy.

The term lower alkylenoxy refers to a lower alkylene substituted at one end by an oxygen atom. Examples of lower alkylenoxy groups are preferably methylenoxy, ethylenoxy and propylenoxy.

The term halogen means fluorine, chlorine, bromine or iodine, preferably fluorine, chlorine and bromine.

The term cycloalkyl alone or in combination, means a saturated cyclic hydrocarbon ring system with 3 to 7 carbon atoms, e.g. cyclopropyl, cyclobutyl, cyclopentyl, cyclohexyl and cycloheptyl, which can be optionally mono- or multisubstituted by lower alkyl, lower alkenyl, lower alkenylene, lower alkoxy, lower alkylenoxy, lower alkylenedioxy, hydroxy, halogen, —CF₃, —NR¹R¹′, —NR¹C(O)R¹′, —NR¹S(O₂)R1′, —C(O)NR¹R¹′, lower alkylcarbonyl, —COOR¹, —SR¹, —SOR¹, —SO₂R¹, —SO₂NR¹R¹′ whereby R¹′ represents hydrogen; lower alkyl; lower alkenyl; lower alkinyl; cycloalkyl; aryl; cycloalkyl-lower alkyl. The cyclopropyl group is a preferred group.

The term aryl, alone or in combination, relates to the phenyl, the naphthyl or the indanyl group, preferably the phenyl group, which can be optionally mono- or multisubstituted by lower alkyl, lower alkenyl, lower alkinyl, lower alkenylene or lower alkylene forming with the aryl ring a five- or six-membered ring, lower alkoxy, lower alkylenedioxy, lower alkylenoxy, hydroxy, hydroxy-lower alkyl, halogen, cyano, —CF₃, —OCF₃, —NR¹′R¹′, —NR¹R¹′-lower alkyl, —NR¹C(O)R¹′, —NR₁S(O₂)R¹, —C(O)NR¹R¹′, —NO₂, lower alkylcarbonyl, —COOR¹, —SR¹, —SOR¹, —SO₂R¹, —SO₂NR¹R¹′, benzyloxy, whereby R¹′ has the meaning given above. Preferred substituents are halogen, lower alkoxy, lower alkyl, CF₃, OCF₃.

The term aryloxy refers to an Ar—O group, wherein Ar is an aryl. An example of a lower aryloxy group is phenoxy.

The term heterocyclyl, alone or in combination, means saturated or unsaturated (but not aromatic) five-, six- or seven-membered rings containing one or two nitrogen, oxygen or sulfur atoms which may be the same or different and which rings can be optionally substituted with lower alkyl, hydroxy, lower alkoxy and halogen. The nitrogen atoms, if present, can be substituted by a —COOR² group. Examples of such rings are piperidinyl, morpholinyl, thiomorpholinyl, piperazinyl, tetrahydropyranyl, dihydropyranyl, 1,4-dioxanyl, pyrrolidinyl, tetrahydrofuranyl, dihydropyrrolyl, imidazolidinyl, dihydropyrazolyl, pyrazolidinyl, dihydroquinolinyl, tetrahydroquinolinyl, tetrahydroisoquinolinyl.

The term heteroaryl, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing one oxygen and one nitrogen atom and benzofused derivatives thereof; five-membered aromatic rings containing a sulfur and a nitrogen or an oxygen atom and benzofused derivatives thereof; five-membered aromatic rings containing two nitrogen atoms and benzofused derivatives thereof; five-membered aromatic rings containing three nitrogen atoms and benzofused derivatives thereof, or a tetrazolyl ring. Examples of such ring systems are furanyl, thiophenyl, pyrrolyl, pyridinyl, pyrimidinyl, indolyl, quinolinyl, isoquinolinyl, imidazolyl, triazinyl, thiazinyl, thiazolyl, isothiazolyl, pyridazinyl, pyrazolyl, oxazolyl, isoxazolyl, coumarinyl, benzothiophenyl, quinazolinyl, quinoxalinyl. Such rings may be adequatly substituted with lower alkyl, lower alkenyl, lower alkinyl, lower alkylene, lower alkenylene, lower alkylenedioxy, lower alkyleneoxy, hydroxy-lower alkyl, lower alkoxy, hydroxy, halogen, cyano, —CF₃, —OCF₃, —NR¹R¹′, —NR¹R¹′-lower alkyl, —N(R¹)COR¹, —N(R¹)SO₂R¹, —CONR¹R¹′, —NO₂, lower alkylcarbonyl, —COOR¹, —SR¹, —SOR¹, —SO₂R¹, —SO₂NR¹R¹′, another aryl, another heteroaryl or another heterocyclyl and the like, whereby R¹′ has the meaning given above. Preferred heteroaryl are pyridinyl, pirimidinyl, pirazinyl.

The term heteroaryloxy refers to a Het-O group, wherein Het is a heteroaryl.

The expression pharmaceutically acceptable salts encompasses either salts with inorganic acids or organic acids like hydrochloric or hydrobromic acid, sulfuric acid, phosphoric acid, citric acid, formic acid, acetic acid, maleic acid, tartaric acid, benzoic acid, methanesulfonic acid, p-toluenesulfonic acid, and the like that are non toxic to living organisms or in case the compound of formula I is acidic in nature with an inorganic base like an alkali or earth alkali base, e.g. sodium hydroxide, potassium hydroxide, calcium hydroxide and the like.

Compounds of the invention also include nitrosated compounds of the general formula I that have been nitrosated through one or more sites such as oxygen (hydroxyl condensation), sulfur (sulffiydryl condensation) and/or nitrogen. The nitrosated compounds of the present invention can be prepared using conventional methods known to one skilled in the art. For example, known methods for nitrosating compounds are described in U.S. Pat. Nos. 5,380,758 and 5,703,073; WO 97/27749; WO 98/19672; WO 98/21193; WO 99/00361 and Oae et al, Org. Prep. Proc. Int., 15(3): 165-198 (1983), the disclosures of each of which are incorporated by reference herein in their entirety.

The compounds of the general formula I can contain two or more asymmetric carbon atoms and may be prepared in form of optically pure enantiomers, mixtures of enantiomers such as racemates, diastereomers, mixtures of diastereomers, diastereomeric racemates, mixtures of diastereomeric racemates, and the meso-form and pharmaceutically acceptable salts thereof.

The present invention encompasses all these forms. Mixtures may be separated in a manner known per se, i.e. by column chromatography, thin layer chromatography, HPLC or crystallization.

A group of preferred compounds of general formula I above are those wherein W, V, and U are as defined in general formula I and

T is —CONR¹—;

Q is methylene;

M is aryl; heteroaryl.

Another group of even more preferred compounds of general formula I are those wherein W, U, T, Q, and M are as defined in general formula I above and

V is —CH₂CH₂O—; —CH₂CH₂CH₂O—; —OCH₂CH₂O—.

Another group of also more preferred compounds of general formula I are those wherein V, U, T, Q, and M are as defined in general formula I above and

W represents a 1,4-disubstituted phenyl group.

Another group of also more preferred compounds of general formula I are those wherein W, V, U, T, Q, and M are as defined in general formula I above and

U is a mono-, di-, or trisubstituted phenyl or heteroaryl, wherein the substituents are halogen, lower alkyl, lower alkoxy, CF₃.

Especially preferred compounds of general formula I are those selected from the group consisting of:

### (rac.)-(1R*, 5S*)-3-{4-[3-(2,3,6-trifluorophenoxy)propyl]phenyl}-8-azabicyclo-[3.2.1]oct-2-ene-2-carboxylic acid cyclopropyl-(2,3-dichlorobenzyl)amide,

### (rac.)-(1R*, 5S*)-3-{4-[3-(2-chloro-3,6-difluorophenoxy)propyl]phenyl}-8-aza-bicyclo[3.2.1]oct-2-ene-2-carboxylic acid cyclopropyl-(2,3-dichlorobenzyl)amide,

### (rac.)-(1R*, 5S*)-3-{4-[3-(4-fluoro-5-methylisoxazol-3-yloxy)propyl]phenyl}-8-azabicyclo[3.2.1]oct-2-ene-2-carboxylic acid cyclopropyl-(3-methoxy-2-methyl-benzyl)amide.

The compounds of general formula I and their pharmaceutically acceptable salts may be used as therapeutics e.g. in form of pharmaceutical compositions. These pharmaceutical compositions containing at least one compound of general formula I and usual carrier materials and adjuvants may especially be used for the treatment or prophylaxis of disorders which are associated with a dysregulation of the renin angiotensin system (RAS), comprising cardiovascular and renal diseases. Examples of such diseases are hypertension, congestive heart failure, pulmonary heart failure, coronary diseases, cardiac insufficiency, renal insufficiency, renal or myocardial ischemia, atherosclerosis, and renal failure. They can also be used to prevent restenosis after balloon or stent angioplasty, to treat erectile dysfunction, glomerulonephritis, renal colic, and glaucoma. Furthermore, they can be used in the therapy and the prophylaxis of diabetic complications, complications after vascular or cardiac surgery, complications of treatment with immunosuppresive agents after organ transplantation, complications of cyclosporin treatment, as well as other diseases presently known to be related to the RAS.

In another embodiment, the invention relates to a method for the treatment and/or prophylaxis of diseases which are related to the RAS such as hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, complications of treatment with immunosuppresive agents after organ transplantation, and other diseases which are related to the RAS, which method comprises administering a compound according of formula I to a human being or animal.

The invention further relates to the use of compounds of general formula I as defined above for the treatment and/or prophylaxis of diseases which are associated with the RAS such as hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, complications of treatment with immunosuppresive agents after organ transplantation, and other diseases presently known to be related to the RAS.

In addition, the invention relates to the use of compounds as defined above for the preparation of medicaments for the treatment and/or prophylaxis of diseases which are associated with the RAS such as hypertension, coronary diseases, cardiac insufficiency, renal insufficiency, renal and myocardial ischemia, and renal failure. These medicaments may be prepared in a manner known per se.

The compounds of formula I may also be used in combination with one or more other pharmacologically active compounds e.g. with other renin inhibitors, with ACE-inhibitors, angiotensin II receptor antagonists, endothelin receptor antagonists, vasodilators, calcium antagonists, potassium activators, diuretics, sympatholitics, beta-adrenergic antagonists, alpha-adrenergic antagonists, and neutral endopeptidase inhibitors, for the treatment of disorders as above-mentioned.

All forms of prodrugs leading to an active component comprised by general formula I above are included in the present invention.

The compounds of general formula I can be manufactured by the methods outlined below, by the methods described in the examples or by analogous methods.

Chemistry

Commercially available tropinone can be acylated racemically or enantioselectively as described in the literature (Majewski, M; et al.; J. Org. Chem., 1995, 60, 5825). Then tropinone derivatives of type A (Scheme 1), whereas R^{b} typically represents a methyl, an ethyl or a benzyl group, may be transformed further accordingly to the chemistry described in earlier patent applications, for instance WO 03/093267 or WO 04/002957. For instance a compound of type A can be converted into a vinyl triflate of type B. A carbon-carbon coupling catalyzed by a metallic complex, like a palladium complex, can lead to a compound of type C, whereas R^{a} represents a substituent that can lead in one or several chemical maipulation to a substituent V-U as described in formula I. R^{a} can be modified during the synthesis. Protecting group manipulations can lead to a compound of type D. Well-known manipupulations at the R^{a}-substituent, like deprotection and Mitsunobu reaction, can lead to a compound of type E. Hydrolysis of the ester can lead to a compound of type F, then amide coupling to a compound of type G. Final deprotection can lead to a desired compound of type H.

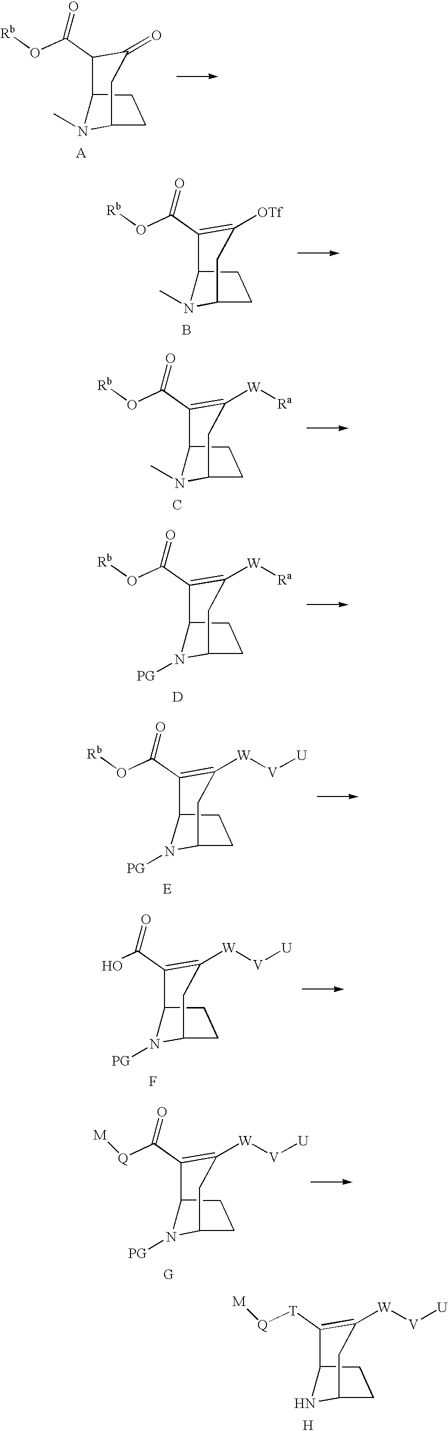

The compounds of formula I and their pharmaceutically acceptable acid addition salts can be used as medicaments, e.g. in the form of pharmaceutical preparations for enteral, parenteral, or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The production of pharmaceutical preparations can be effected in a manner which will be familiar to any person skilled in the art by bringing the described compounds of formula I and their pharmaceutically acceptable acid addition salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injections are, for example, water, alcohols, polyols, glycerols and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of compounds of formula I can vary within wide limits depending on the disease to be controlled, the age and the individual condition of the patient and the mode of administration, and will, of course, be fitted to the individual requirements in each particular case. For adult patients a daily dosage of about 1 mg to about 1000 mg, especially about 50 mg to about 500 mg, comes into consideration.

The pharmaceutical preparations conveniently contain about 1-500 mg, preferably 5-200 mg of a compound of formula I.

The following examples serve to illustrate the present invention in more detail. They are, however, not intended to limit its scope in any manner.

### EXAMPLES

### Abbreviations

ACE Angiotensin Converting Enzyme
Ang Angiotensin
aq. aqueous
Boc tert-Butyloxycarbonyl
BSA Bovine serum albumine
BuLi n-Butyllithium
DIPEA Diisopropylethylamine
DMAP 4-N,N-Dimethylaminopyridine
DMSO Dimethylsulfoxide
EDC.HCl Ethyl-N,N-dimethylaminopropylcarbodiimide hydrochloride
EIA Enzyme immunoassay
eq. equivalent
Et Ethyl
EtOAc Ethyl acetate
FC Flash Chromatography
HOBt Hydroxybenzotriazol
MeOH Methanol
NMO N-Methylmorpholine N-oxide
org. organic
PG protecting group
Ph Phenyl
RAS Renin Angiotensin System
rt room temperature
sol. Solution
TBDMS tert-Butyldimethylsilyl
Tf Trifluoromethylsulfonyl
THF Tetrahydrofuran
   Precursors

### (rac.)-(1R*, 5S*)-8-Methyl-3-trifluoromethanesulfonyloxy-8-azabicyclo-[3.2.1]oct-2-ene-2-carboxylic acid methyl ester (B)

A sol. of compound A (1.81 g, 9.12 mmol) in THF (35 mL) was cooled to 0° C. and NaH (about 60% in mineral oil, 435 mg, about 10.0 mmol) was added. A gas evolution was observed. After 20 min, Tf₂NPh (3.86 g, 10.8 mmol) was added. 10 min later, the ice bath was removed. The sol. was stirred overnight, and diluted with EtOAc and washed with brine (1×). The org. extracts were dried over MgSO₄, filtered, and the solvents were removed under reduced pressure. Purification by FC yielded the title compound (2.37 g, 78%).

### (rac.)-(1R*, 5S*)-3-{4-[3-(tert-Butyldimethylsilanyloxy)propyl]phenyl}-8-methyl-8-azabicyclo[3.2.1]oct-2-ene-2-carboxylic acid methyl ester (C)

A sol. of [3-(4-bromophenyl)propoxy]-tert-butyldimethylsilane (Kiesewetter D. O., Tetrahedron Asymmetry, 1993, 4, 2183, 16.47 g, 50.0 mmol) in THF (250 mL) was cooled to −78° C. BuLi (1.6M in hexane, 31.0 mL, 50.0 mmol) was added. After 30 min, ZnCl₂ (1M in THF, 52 mL, 52 mmol, prepared from ZnCl₂ dried overnight at 150° C. and THF) was added. The mixture was allowed to warm up to rt. Vinyl triflate B (7.90 g, 24.0 mmol) in THF (20 mL) and then Pd(PPh₃)₄ (500 mg, 0.43 mmol) were added. The mixture was heated tro reflux for 90 min and aq. 1M HCl (1 mL) was added. The mixture was diluted with EtOAc and washed with aq. 1M NaOH (1×). The org. extracts were dried over MgSO₄, filtered and the solvents were removed under reduced pressure. Purification of the residue by FC yielded the title product (8.44 g, 82%).

### (rac.)-(1R*, 5S*)-3-[4-(3-Hydroxypropyl)phenyl]-8-azabicyclo[3.2.1]oct-2-ene-2,8-dicarboxylic acid 8-tert-butyl ester 2-methyl ester (D)

1-Chloroethyl chloroformate (7.98 g, 56.0 mmol) was added to a sol. of bicycloctene C (8.07 g, 18.8 mmol) in 1,2-dichloroethane (120 mL). The sol. was heated to reflux. After 4 h, the reaction mixture was allowed to cool to rt, and the solvents were removed under reduced pressure. MeOH (100 mL) was added. The mixture was stirred at 75° C. for 30 min, and the solvents were removed under reduced pressure. The residue was diluted with EtOAc and washed with aq. 1M NaOH (2×). The org. extracts were dried over MgSO₄, filtered, and the solvents were removed under reduced pressure. The residue was dissoled in CH₂Cl₂ (50 mL), DIPEA (4.70 g, 36.0 mmol) was added, and the mixture was cooled to 0° C. Boc₂O (4.65 g, 21.0 mmol) was added and the mixture was stirred at 0° C. for 1 h, then at rt for 2 h. The mixture was washed with aq. 1M HCl (1×), and aq. sat. NaHCO₃ (1×). The org. extracts were dried over MgSO₄, filtered, and the solvents were removed under reduced pressure. Purification of the residue by FC yielded the title compound (4.81 g, 64%).

### (rac.)-(1R*, 5S*)-3-{4-[3-(2,3,6-Trifluorophenoxy)propyl]phenyl}-8-aza-bicyclo[3.2.1]oct-2-ene-2,8-dicarboxylic acid 8-tert-butyl ester 2-methyl ester (E1)

Tributylphosphine (3.18 g, 14.0 mmol) was added to a sol. of bicycloctene D (2.09 g, 5.2 mmol), 2,3,6-trifluorophenol (1.59 g, 10.7 mmol) and azodicarboxylic dipiperidide (2.70 g, 10.7 mmol) in toluene (50 mL). The mixture was heated to reflux for 2 h and allowed to cool to rt. The solvents were removed under reduced pressure. Purification by FC yielded the title compound (2.15 g, 78%).

### (rac.)-(1R*, 5S*)-3-{4-[3-(2-Chloro-3,6-difluorophenoxy)propyl]phenyl}-8-azabicyclo[3.2.1]oct-2-ene-2,8-dicarboxylic acid 8-tert-butyl ester 2-methyl ester (E2)

Tributylphosphine (1.61 mL, 7.2 mmol) was added to a sol. of bicycloctene D (1.04 g, 2.59 mmol), 2-chloro-3,6-trifluorophenol (833 mg, 5.10 mmol) and azodicarboxylic dipiperidide (1.29 g, 5.10 mmol) in toluene (25 mL). The mixture was heated to reflux for 2 h and allowed to cool to rt. The solvents were removed under reduced pressure. Purification by FC yielded the title compound (1.11 g, 78%).

### (rac.)-(1R*, 5S*)-3-{4-[3-(4-Fluoro-5-methylisoxazol-3-yloxy)propyl]phenyl}-8-azabicyclo[3.2.1]oct-2-ene-2,8-dicarboxylic acid 8-tert-butyl ester 2-methyl ester (E3)

Tributylphosphine (13.72 mL, 47.4 mmol) was added to a sol. of bicycloctene D (6.34 g, 15.8 mmol), 4-fluoro-5-methylisoxazol-3-ol (Nakayama, E.; Watanabe, K.; Miyauchi, M.; Fujimoto, K.; Ide, J. of Antbiotics, 1990, 43, 1122, 2.77, 23.7 mmol) and azodicarboxylic dipiperidide (5.98 g, 31.6 mmol) in toluene (25 mL). The mixture was heated to reflux for 2 h and allowed to cool to rt. The solvents were removed under reduced pressure. Purification by FC yielded the title compound (6.05 g, 76%).

### (rac.)-(1R*, 5S*)-3-{4-[3-(2,3,6-trifluorophenoxy)propyl]phenyl}-8-aza-bicyclo[3.2.1]oct-2-ene-2,8-dicarboxylic acid 8-tert-butyl ester (F1)

Bicycloctene E1 (1.75 g, 3.29 mmol) was dissolved in EtOH (30 mL). Aq. 1M NaOH (30 mL) was added and the mixture was heated to 80° C. The sol. was stirred for 5 h at 80° C., then allowed to cool down to rt. After acidification to pH=1-2 with aq. 1M HCl the mixture was extracted with EtOAc (3×). The combined org. extracts were dried over MgSO₄, filtered and the solvents were removed under reduced pressure. Purification of the residue by FC yielded the title compound (1.78 g, quantitative).

### (rac.)-(1R*, 5S*)-3-{4-[3-(2-Chloro-3,6difluorophenoxy)propyl]phenyl}-8-azabicyclo[3.2.1]oct-2-ene-2,8-dicarboxylic acid 8-tert-butyl ester (F2)

Bicycloctene E2 (2.42 g, 4.40 mmol) was dissolved in EtOH (50 mL). Aq. 1M NaOH (40 mL) was added and the mixture was heated to 80° C. The sol. was stirred for 5 h at 80° C., then allowed to cool down to rt. After acidification to pH=1-2 with aq. 1M HCl the mixture was extracted with EtOAc (3×). The combined org. extracts were dried over MgSO₄, filtered and the solvents were removed under reduced pressure. Purification of the residue by FC yielded the title compound (2.48 g, quantitative).

### (rac)-(1R*, 5S*)-3-{4-[3-(4-Fluoro-5-methylisoxazol-3-yloxy)propyl]phenyl}-8-azabicyclo[3.2.1]oct-2-ene-2,8-dicarboxylic acid 8-tert-butyl ester (F3)

Bicycloctene E3 (6.05 g, 12.08 mmol) was dissolved in EtOH (115 mL). Aq. 1M NaOH (90 mL) was added and the mixture was heated to 80° C. The sol. was stirred for 5 h at 80° C., then allowed to cool down to rt. After acidification to pH=1-2 with aq. 1M HCl the mixture was extracted with EtOAc (3×). The combined org. extracts were dried over MgSO₄, filtered and the solvents were removed under reduced pressure. Purification of the residue by FC yielded the title compound (4.98 g, 84%).

### EXAMPLES

### Example 1

### (rac.)-(1R*, 5S*)-3-{4-[3-(2,3,6-Trifluorophenoxy)propyl]phenyl}-8-aza-bicyclo[3.2.1]oct-2-ene-2-carboxylic acid cyclopropyl-(2,3-dichlorobenzyl)-amide

A mixture of bicyclononene F1 (0.89 g, 1.70 mmol), cyclopropyl-(2,3-dichloro-benzyl)amine (1.08 g, 5.00 mmol), DIPEA (0.87 gL, 6.70 mmol), DMAP (60 mg, 0.50 mmol), HOBt (137 mg, 1.00 mmol) and EDC.HCl (0.96 g, 5.00 mmol) in CH₂Cl₂ (10 mL) was stirred at rt for 3 days. The mixture was diluted with more CH₂Cl₂, and washed with aq. 1M HCl (3×) and aq. sat. NaHCO₃ (1×). The org. extracts were dried over MgSO₄, filtered, and the solvents were removed under reduced pressure. Purification of the residue by FC yielded the intermediate compound. This intermediate compound was diluted with CH₂Cl₂ (10 mL) and the mixture was cooled to 0° C. HCl (4M in dioxane, 10 mL) was added and the mixture was stirred for 1 h at 0° C., then 1 h at rt. The solvents were removed under reduced pressdure and the residue was dried under high vacuum. The residue was diluted with CH₂Cl₂ and washed with aq. 1M NaOH until the org. phase had a pH>9. The org. extracts wer dried over MgSO₄, filtered, and the solvents were removed under reduced pressure. Purification of the residue by FC yielded the title compound (497 mg).

### Example 2

### (rac.)-(1R*, 5S*)-3-{4-[3(2-Chloro-3,6-difluorophenoxy)propyl]phenyl}-8-azabicyclo[3.2.1]oct-2-ene-2-carboxylic acid cyclopropyl-(2,3-dichloro-benzyl)amide

A mixture of bicyclononene F2 (2.01 g, 3.70 mmol), cyclopropyl-(2,3-dichloro-benzyl)amine (2.43 g, 11.2 mmol), DIPEA (2.07 g, 16.0 mmol), DMAP (135 mg, 1.10 mmol), HOBt (486 mg, 3.60 mmol) and EDC.HCl (2.49 g, 13.0 mmol) in CH₂Cl₂ (30 mL) was stirred at rt for 3 days. The mixture was diluted with more CH₂Cl₂, and washed with aq. 1M HCl (3×) and aq. sat. NaHCO₃ (1×). The org. extracts were dried over MgSO₄, filtered, and the solvents were removed under reduced pressure. Purification of the residue by FC yielded the intermediate compound. This intermediate compound was diluted with CH₂Cl₂ (10 mL) and the mixture was cooled to 0° C. HCl (4M in dioxane, 10 mL) was added and the mixture was stirred for 1 h at 0° C., then 1 h at rt. The solvents were removed under reduced pressdure and the residue was dried under high vacuum. The residue was diluted with CH₂Cl₂ and washed with aq. 1M NaOH until the org. phase had a pH>9. The org. extracts wer dried over MgSO₄, filtered, and the solvents were removed under reduced pressure. Purification of the residue by FC yielded the title compound (1.35 g).

### Example 3

### (rac.)-(1R*, 5S*)-3-{4-[3-(4-Fluoro-5-methylisoxazol-3-yloxy)propyl]phenyl}-8-azabicyclo[3.2.1]oct-2-ene-2-carboxylic acid cyclopropyl-(3-methoxy-2-methylbenzyl)amide

A mixture of bicyclononene F3 (2.50 g, 5.14 mmol), cyclopropyl-(2-methyl-3-methoxybenzyl)amine (prepared by reductive amination from 3-methoxy-2-methylbenzaldehyde, Comins, D. L.; Brown, J. D., J. Org. Chem., 1989, 54, 3730, and cyclopropylamine; 2.95 g, 15.4 mmol), DIPEA (3.52 mL, 20.6 mmol), DMAP (157 mg, 1.29 mmol), HOBt (903 mg, 6.69 mmol) and EDC.HCl (2.47 g, 12.9 mmol) in CH₂Cl₂ (30 mL) was stirred at rt for 3 days. The mixture was diluted with more CH₂Cl₂, and washed with aq. 1M HCl (3×) and aq. sat. NaHCO₃ (1×). The org. extracts were dried over MgSO₄, filtered, and the solvents were removed under reduced pressure. Purification of the residue by FC yielded the intermediate compound. This intermediate compound was diluted with CH₂Cl₂ (10 mL) and the mixture was cooled to 0° C. HCl (4M in dioxane, 10 mL) was added and the mixture was stirred for 1 h at 0° C., then 1 h at rt. The solvents were removed under reduced pressdure and the residue was dried under high vacuum. The residue was diluted with CH₂Cl₂ and washed with aq. 1M NaOH until the org. phase had a pH>9. The org. extracts wer dried over MgSO₄, filtered, and the solvents were removed under reduced pressure. Purification of the residue by FC yielded the title compound (1.54 g).

Inhibition of Human Recombinant Renin by the Compounds of the Invention

The enzymatic in vitro assay was performed in 384-well polypropylene plates (Nunc). The assay buffer consisted of 10 mM PBS (Gibco BRL) including 1 mM EDTA and 0.1% BSA. The incubates were composed of 50 μL per well of an enzyme mix and 2.5 μL of renin inhibitors in DMSO. The enzyme mix was premixed at 4° C. and consists of the following components:

human recombinant renin (0.16 ng/mL)•synthetic human angiotensin(1-14) (0.5 μM)

hydroxyquinoline sulfate (1 mM)

The mixtures were then incubated at 37° C. for 3 h.

To determine the enzymatic activity and its inhibition, the accumulated Ang I was detected by an enzyme immunoassay (EIA) in 384-well plates (Nunc). 5 μL of the incubates or standards were transferred to immuno plates which were previously coated with a covalent complex of Ang I and bovine serum albumin (Ang I-BSA). 75 μL of Ang I-antibodies in essaybuffer above including 0.01% Tween 20 were added and a primary incubation made at 4° C. overnight. The plates were washed 3 times with PBS including 0.01% Tween 20, and then incubated for 2 h at rt with an antirabbit-peroxidase coupled antibody (WA 934, Amersham). After washing the plates 3 times, the peroxidase substrate ABTS (2.2′-azino-di-(3-ethyl-benzthiazolinsulfonate), was added and the plates incubated for 60 min at room temperature. After stopping the reaction with 0.1 M citric acid pH 4.3 the plate was evaluated in a microplate reader at 405 nm. The percentage of inhibition was calculated of each concentration point and the concentration of renin inhibition was determined that inhibited the enzyme activity by 50% (IC₅₀). The IC₅₀-values of all compounds tested are below 100 nM. However selected compounds exhibit a very good bioavailibility and are metabolically more stable than prior art compounds.

1. A compound of formula I 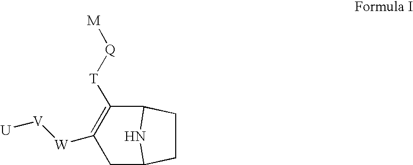
wherein
W is a six-membered, non benzofused, phenyl or heteroaryl ring, substituted by V in meta or para position;
V represents a bond; —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂CH₂CH₂-A-CH₂—CH₂—B—CH₂—; —CH₂-A-CH₂—CH₂—CH₂—B—; —CH₂—CH₂-A-CH₂—CH₂—B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O—; or —O—C(CH₂CH₂)—CH₂—O—;
A and B independently represent —O—; —S—; —SO—; or —SO₂—;
U represents aryl; or heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; or lower alkenylene;
M represents hydrogen; cycloalkyl; aryl; heterocyclyl; or heteroaryl;
R¹ represents hydrogen; lower alkyl; lower alkenyl; lower alkinyl; cycloalkyl; aryl; or cycloalkyl-lower alkyl;
p is the integer 1, 2, 3 or 4;
r is the integer 3, 4, 5, or 6;
s is the integer 2, 3, 4, or 5;
t is the integer 1, 2, 3, or 4;
u is the integer 1, 2, or 3; and
v is the integer 2, 3, or 4;
or optically pure enantiomers, racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

2. The compound of formula I according to claim 1, wherein
T represents —CONR¹—;
Q represents methylene; and
M represents aryl; or heteroaryl;
or optically pure enantiomers, racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

3. The compound of formula I according to claim 1, wherein
V represents —CH₂CH₂O—; —CH₂CH₂CH₂O—; or —OCH₂CH₂O—;
or optically pure enantiomers, racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

4. The compound of formula I according to claim 1, wherein
W represents a 1,4-disubstituted phenyl group;
or optically pure enantiomers, racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

5. The compound of formula I according to claim 1, wherein
U is a mono-, di-, or trisubstituted phenyl or heteroaryl, whereby the substituents are selected from the group consisting of halogen, lower alkyl, lower alkoxy, and CF₃
or optically pure enantiomers, racemates, diastereomers, mixtures of diastereomers, diastereomeric racemates, mixtures of diastereomeric racemates, or the meso-form of the compound; or pharmaceutically acceptable salts, solvent complexes or morphological forms of the compound.

6. The compound according to claim 1 selected from the group consisting of:
(rac.)-(1R*, 5S*)-3-{4-[3-(2,3,6-trifluorophenoxy)propyl]phenyl}-8-azabicyclo-[3.2.1]oct-2-ene-2-carboxylic acid cyclopropyl-(2,3-dichlorobenzyl)amide,
(rac.)-(1R*, 5S*)-3-{4-[3-(2-chloro-3,6-difluorophenoxy)propyl]phenyl}-8-aza-bicyclo[3.2.1]oct-2-ene-2-carboxylic acid cyclopropyl-(2,3-dichlorobenzyl)amide, and
(rac.)-(1R*, 5S*)-3-{4-[3-(4-fluoro-5-methylisoxazol-3-yloxy)propyl]phenyl}-8-azabicyclo[3.2.1]oct-2-ene-2-carboxylic acid cyclopropyl-(3-methoxy-2-methyl-benzyl)amide.

7. A pharmaceutical composition comprising at least one compound of claim 1 and a carrier and/or an adjuvant.

8. A method for the treatment or prophylaxis of RAS-associated diseases comprising hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, or complications of treatment with immunosuppressive agents after organ transplantation, which method comprises administering a compound according to claim 1 to a human being or animal.

9. (canceled)

10. (canceled)
